(19) United States
(12) Patent Application Publication
(10) Pub. No.: US 2011/0077573 A1
(43) Pub. Date: Mar. 31, 2011

(54) Protective Wraps and Methods of Animal Wound Care

(76) Inventor: Karen Pain, Austin (CA)

(21) Appl. No.: 12/568,296

(22) Filed: Sep. 28, 2009

(51) Int. Cl. A61F 13/06; A61F 13/00

(52) U.S. Cl. 602/63; 602/79

(57) ABSTRACT

A wound wrap system for an animal features a flexible wrap with a cover layer and an absorbent liner layer. Flexible straps at one or both side edges of the wrap are used in belt-like connections to loop around a body part of the animal to secure the absorbent material of the wrap over a wounded area of the body part. In embodiments for use on an animal's torso or front leg, an additional strap arrangement at an end edge lies perpendicular to the other straps when the wrap and straps are laid out flat and is extendable in a connection across a back of the animal's neck in use of the wrap. Absorbent pads or inserts are selectively securable against the wrap's liner for use in early stages of wound recovery, where leakage is more prominent, and subsequently removable for later healing stages. All components are washable for re-use.

### FIELD OF THE INVENTION

The present invention relates generally to a protective wrapping system for care of wounds for animals, and more particularly to protective wraps intended for use with longer term wound management in the home of a pet owner.

### BACKGROUND OF THE INVENTION

Wound care for small animals is a particular challenge given the proclivity of animals for licking their wounds, rolling in dirt, and needing relatively free range of motion. Long term care of animal wounds (from several weeks through several months) is especially challenging because pet owners need bandaging systems that are inexpensive, easy to change, and are tolerated well by pets. The bandaging systems also need to be highly effective since many pets now reside in the homes of their owners, and the problem of fluid leakage around the home is a major concern (destroying carpets, flooring, etc.). Indeed many pet owners decide to euthanize their animals because the pet's long term care of wounds would damage the home environment.

Previous efforts in this general field are summarized below with reference to U.S. patent numbers.

Many inventors have attempted to address challenges of wrapping or bandaging small animal wounds on various locations of the animal's body. Torso wound protection has been addressed by proposing: a head restraint that limits head motion to prevent licking of wounds (U.S. Pat. No. 7,458,339), a canine cervical jacket that restricts range of motion (U.S. Pat. No. 4,489,676), an elastic wound dressing that self-adheres to an absorbent layer that attaches to the animal (U.S. Pat. No. 5,939,339), a coat that secures a bandage to the body of the animal (U.S. Pat. No. 3,895,628), a protective garment for post-surgery recovery (U.S. Pat. No. 6,234,117), using disposable protective wrapping bandages (U.S. Pat. No. 5,137,508) and using an elastic-like tube that stretches to fit around the body of the animal (U.S. Pat. No. 4,355,600). Shesol (U.S. Pat. No. 7,004,922) proposed an animal wound wrap for holding a primary dressing which also has attachments for drains and IV tubes. Hamada (U.S. Pat. No. 6,454,735) outlines a simple bandage for use on an animal's torso (chest area or abdomen) which stretches to fit around the animal.

Ideas for protective small-animal leg wraps have also evolved over the years. They range from a wound shield which is a plastic cone to protect a bandage (U.S. Pat. No. 7,185,612), to protective wraps designed to shield a dog's leg from insects and burrs (U.S. Pat. No. 5,676,094), to leg wraps which have sleeves in them to hold ice packs for swelling (U.S. Pat. No. 6,240,882), and a leg wrap with cushioning pillows through several layers of protection (U.S. Pat. No. 6,883,466). There have been many designs for leg wraps that wrap around dog's legs for protection (U.S. Pat. No. 6,564,753), which include padding to address elbow ulcer issues (U.S. Pat. Nos. 5,341,765 and 5,076,043). U.S. Pat. No. 4,479,457 describes an elbow pad that fits around the elbow of the dog, can accommodate an adhesive pad, and is specifically shaped to cover the elbow.

Providing wound coverage for feet/paws is especially challenging since the protective wrapping needs to stay on the animal through a range of motion. Many patents have addressed foot injuries in horses: U.S. Pat. No. 6,883,466 discloses a padded wrap, U.S. Pat. No. 5,528,885 proposes a foot cover that laces up the horse's leg and U.S. Pat. No. 5,224,549 presents a boot type shoe for horses. U.S. Pat. No. 3,762,073 proposes plastic, disposable coverings for dog's legs. A set of booties that fit inside each other and strap around the collar of a dog have been described in U.S. Pat. No. 5,676,095, and a bootie suspender system has been developed (U.S. Pat. No. 4,744,333). Protective sleeves for leg casts for outdoor use have been proposed (U.S. Pat. No. 5,452,685). U.S. Pat. No. 6,883,466 describes a protection sock/shoe for a dog's paw that can act as a bandage, while U.S. Pat. No. 6,186,097 describes a protection shoe for dogs which has an outer waterproof layer.

Despite all these efforts, there remains room for improvement in the art.

### SUMMARY OF THE INVENTION

According to a first aspect of the invention there is provided a wound wrap system for an animal comprising:

a flexible wrap comprising a cover layer of a first material and a liner layer fixed to the cover layer and comprising an absorbent material different than the first material;

a set of flexible straps each having a first connection to the flexible wrap and each having a distal end spaced from and movable relative to the first connection, each strap carrying a fastening element thereon between the first connection and the distal end, each fastening element being selectively fastenable with a mating fastening element carried on the flexible wrap to close a loop around a body part of the animal to secure the absorbent material of the liner layer of the flexible wrap over a wounded area of the body part.

Each flexible strap may extend from the wrap at a side edge thereof and the wound wrap system further comprises an additional strap arrangement extending from the wrap at an end edge thereof so that the flexible straps and the additional strap arrangement project from the wrap along opposing ones of perpendicular axes in a plane of the wrap when laid out flat. The additional strap arrangement preferably comprises a pair of straps extending from the end edge of the wrap.

The wrap may be sized to wrap about at least a portion of an animal's torso, each flexible strap being sufficiently long to close the loop around the animal's torso and the additional strap arrangement being sufficiently long to secure about an animal's neck.

Alternatively, the wrap may be sized to wrap about at least a portion of an animal's leg, each flexible strap being sufficiently long to close the loop around the animal's leg.

As a further alternative, the wrap may be configured into an elongated substantially tubular configuration having an open end and an opposing closed end, with the liner layer of the absorbent material disposed inside the substantially tubular configuration.

Preferably a longitudinal opening in one side of the substantially tubular configuration extends from the open end thereof toward, without reaching, the opposing closed end, a portion of the substantially tubular configuration along which the longitudinal opening extends defining a leg portion for receipt of an animal's leg and a remaining portion of the substantially tubular configuration between the longitudinal opening and the closed end defining a foot portion for receipt of an animal's foot. In this instance, at least some of the flexible straps are preferably connected to the substantially tubular configuration at positions along the leg portion thereof.

Preferably a drawstring is disposed about the substantially tubular configuration of the wrap adjacent the open end thereof and operable to reduce a size of the open end.

Preferably there is provided a gripping element situated externally on the tubular configuration of the wrap adjacent the closed end thereof.

Preferably there is provided an absorbent tubular insert sized for receipt within the substantially tubular configuration of the wrap and being open at one end and closed at an opposing end.

Preferably the absorbent tubular liner is made of the same absorbent material as the liner layer of the wrap.

Preferably the wrap includes at least one additional absorbent layer between the cover layer and the liner layer.

Preferably the at least one additional absorbent layer comprises the same absorbent material as the liner layer.

In embodiments where the wrap is not configured into a tubular configuration, preferably there is provided at least one absorbent pad of similar shape and size to the wrap and selectively attachable and detachable to the liner layer of the wound wrap system.

Preferably the liner layer comprises cotton flannelette.

According to a second aspect of the invention there is provided a method of animal wound care comprising the steps of:

(a) providing a wound wrap system comprising:
a flexible wrap comprising at least one layer of absorbent material;
a set of flexible straps each extendable from a side edge of the wrap; and
an additional strap arrangement extending from the wrap at an end edge thereof so that the flexible straps and the additional strap arrangement project from the wrap along opposing ones of perpendicular axes in a plane of the wrap when laid out flat;

(b) using the flexible strap to close loops around a torso or front leg of an animal to secure the absorbent material of the flexible wrap over a wounded area of the torso or front leg; and

(c) extending portions of the additional strap arrangement about opposite sides of a neck of the animal to interconnect the portions of the additional strap arrangement across a back of the neck to provide additional support of the wrap on the torso or front leg.

According to a third aspect of the invention there is provided method of animal wound care comprising the steps of:

(a) providing a wound wrap system comprising:
a flexible wrap comprising a cover layer of a first material and a liner layer fixed to the cover layer and comprising an absorbent material different than the first material; and
an absorbent pad of similar shape and size to the wrap and selectively securable against the liner layer of the wound wrap system;

(b) with the absorbent pad secured against the liner layer of the wound wrap system, securing the wrap about at least a portion of a body part of the animal with the absorbent pad facing the body part and covering a wounded area thereof;

(c) keeping the wrap and the absorbent pad on the animal for a period of time with the wounded area being protected by the wrap and the absorbent pad during said period of time;

(d) removing the wrap from the animal and removing the absorbent pad from the wrap;

(e) securing the wrap at least partially about the body part of the animal with the liner layer of the wrap facing toward the body part to again cover the wounded area; and

(f) washing the absorbent pad while the wrap is worn by the animal to prepare the absorbent pad for later re-use if needed.

In this method, preferably the wound wrap system further comprises an additional absorbent pad other than that removed in step (d), but also of similar shape and size to the wrap and selectively securable against the liner layer of the wound wrap system, and depending on a desired degree of absorbency based on a status of the wound discovered in step (d), step (e) is carried out either (i) with the additional absorbent pad secured against the liner layer of the wrap or (ii) with no separate absorbent pad secured against the wrap so that the absorbent layer of the wrap is placed directly over the wounded area of the animal.

### BRIEF DESCRIPTION OF THE DRAWINGS

In the accompanying drawings, which illustrate a exemplary embodiments of the present invention:

FIG. 1 schematically illustrates a body wrap system according to a first embodiment of the present invention covering a wound on a dog's stomach.

FIG. 2 is a laid out plan view of a body wrap of the first embodiment system.

FIG. 3 is a laid out plan view of a removable pad of the first embodiment system.

FIG. 4 schematically illustrates a leg wrap system according to a second embodiment of the present invention worn on a front leg of a dog.

FIG. 5 is a laid out plan view of a leg wrap of the second embodiment system.

FIG. 6 is a laid out plan view of the leg wrap of FIG. 5 from an opposite side thereof.

FIG. 7 is a laid out plan view of a removable pad of the second embodiment system.

FIG. 8 schematically illustrates a foot wrap system according to a third embodiment of the present invention worn on a rear leg of a dog.

FIG. 9 is a front elevational view of a foot wrap of the third embodiment system.

FIG. 10 is a rear elevational view of the foot wrap of the third embodiment system.

FIG. 11 is an elevational view of a sock-like insert or pad of the third embodiment system.

### DETAILED DESCRIPTION

### Body or Torso Protective Wrap System

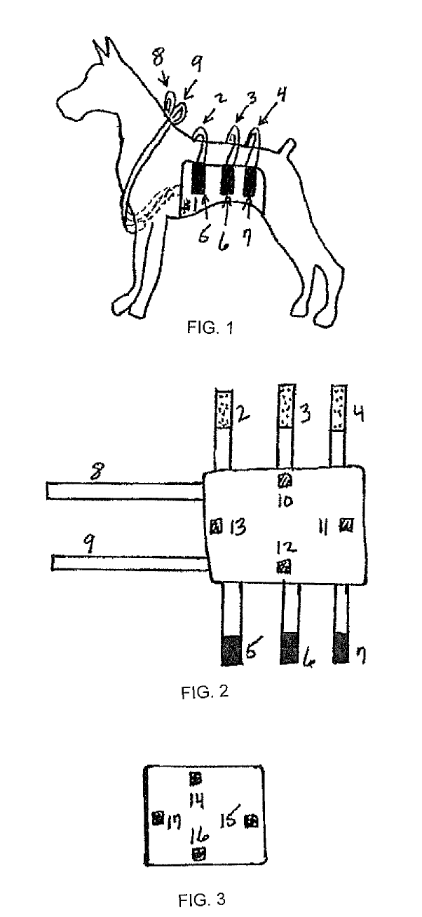

FIGS. 1 and 2 illustrate a torso protective wrap 1 having a main central portion that is rectangular in shape when laid out flat and has a multiple layer configuration with an outer layer of polyester fleece with an inner lining consisting of two layers of cotton flannelette. The inner lining is sewn or otherwise attached to the outer fleece layer so as to form an intentionally permanent part of the protective garment that is not to be removed. The rectangular shape of the central portion is elongated in one direction so that this piece may be considered to have two longitudinal sides and two transverse ends, respectively oriented horizontally and vertically on the page in FIG. 2. At spaced positions along each longitudinal side are sewn three fastening belts. Each belt is positioned equidistant from the next belt on the same side of the central piece so the three belts are respectively attached near the middle and each end of the respective side. Accordingly, each strap of the belt aligns with a respective belt strap on the opposite side across the central piece. The fastening belt straps 2, 3, 4 on one side of the central piece have hook tape fixed thereon, while the belt straps 5, 6, 7 on the opposite have loop tape fixed thereon, the result being three straps on one side having hook fastener elements and the other three straps on the opposing side having loop fastener elements. On each belt strap, the hook or loop tape is secured on both sides of the strap so that each strap can be fastened to a respective strap extending from the opposite side regardless of which of the two straps is folded over the other. On each strap, the hook or loop tape extends from the distal end of the strap back toward the central piece.

The inner lining of the torso protective wrap is on the side or face of the central piece to be placed against the body of the animal in use of the wrap, and is accordingly made from two layers of cotton flannelette to absorb fluid leakage from wounds and to provide cushioning around the wound. With the torso protective wrap having an outer shell made from polyester fleece and an inner lining made from cotton flannelette, the wrap is designed to withstand multiple cleanings, by hand or machine washing and air or machine drying, to allow long term and repeated use of the wrap. Compared to use of disposable bandages, the washable reusable wrap is environmentally friendly and can be more cost efficient in use in healing of different wounds over time.

FIG. 1 shows use of the wrap in covering a stomach or belly wound on a dog. The body wrap 1 is placed on the stomach and the two sides are wrapped upward about each side of the dog so, the wrap covering approximately ½ the body of the animal. The belt straps are then pulled around each side of the animal and fastened together in pairs, so that the animal is encased in the body wrap. That is, the hook-equipped fastening straps 2, 3, and 4 lap over the opposing three loop-equipped fastening straps attached to the other side of the central wrap piece over the dog's back to secure the wrap to the dog. Two tying straps are sewn to the central piece of the wrap adjacent one of the transverse ends thereof, and they can be tied around the neck of the animal, attached to a collar worn by the animal, or otherwise engaged about the animal at or proximate the neck. The neck ties 8,9 are placed equidistant across the front end of the wrap and are quite long, for example each about 24 inches in length, so they can be passed forwardly between the dog's two front legs and be wrapped and tied around the dog's neck in a bow at the base of the neck or fastened to the collar.

Because there is no neck piece on the body wrap, the same procedures can be followed to fasten the body wrap on a back wound by placing the wrap on the back of the animal, and securing the hook and loop fastener belts around the stomach. In like manner the body wrap can be used to protect wounds on either side of the body by placing the wrap on the side of the dog, and bringing the hook and loop fasteners around the back and the stomach, fastening them together on the opposite side of the animal.

There are four small pieces of loop fastener material sewn into the inner lining proximate respective sides of the central piece of the wrap, each piece for example being approximately 1½ inches in from the respective side of the torso protective wrap. In addition to the wrap itself, each torso protective wrap system preferably includes two removeable cotton pads, one of which is shown in FIG. 3 laid out flat on a horizontal surface. Each pad is preferably made from three layers of cotton flannelette and is rectangular in shape and slightly smaller than the central piece of the wrap, for example about 1 inch smaller than the wrap in each of its two dimensions (length and width) when laid out fiat. On one side of the pad are sewn four small pieces of hook fastener material, which are positioned on the pad adjacent the four sides thereof at generally central positions the sides in order to line up with the fastener material pieces on the central portion of the wrap when the pad is centered thereover. The four loop fastener elements 10, 11, 12, 13 sewn to the lining of the wrap to thus fasten to the corresponding hook fastener elements 14, 15, 16, 17 on the pad to selectively receive and secure the one of the two extra pads on the wrap. Each pad can thus be quickly and easily attached to the main body wrap to prevent slippage between the wrap and the pad when used on the animal.

### Leg Protective Wrap System

A leg protective wrap system following similar design features is described next. Shown in isolation laid out on a horizontal surface in FIG. 5, the leg protective wrap has a main central portion that is rectangular in shape and constructed out of polyester fleece with an inner lining consisting of two layers of cotton flannelette. The inner lining is part sewn or otherwise attached to the outer fleece layer so as to form an intentionally permanent part of the protective garment that is not to be removed. The rectangular shape of the central portion is elongated in one direction so that this piece may be considered to have two longitudinal sides and two transverse ends, respectively oriented vertically and horizontally on the page in FIG. 5. At spaced positions along one longitudinal side of the central portion are sewn three fastening belts or straps. Each belt is positioned equidistant from the others so the three belts are respectively attached near the middle and each end of the respective side of the central portion. Three strips of loop fastener material fixed to the outer fleece side of the central piece extend thereacross parallel to the transverse ends thereof at positions along the longitudinal dimension of the central piece aligning with where the belts are fixed thereto. Each belt has a strip of hook fastener material fixed to the side thereof that faces the direction opposite the strips of loop fastener on the central piece when the entire wrap is laid out flat, as it is in FIG. 5. FIG. 5 shows the leg wrap with the three hook fastener belt closure straps sewn near the side of the wrap. In application of the wrap, these closures 114, 115, 116 circle around the wrap having been placed around the animal's leg, and can then be pulled to tighten around the leg and fastened to the three loop fastener closure strips 117, 118, 119 which extend across the front of the wrap's outer fleece shell. The loop fastener strips preferably cover all, or nearly all, of the wrap's width so that the wrap can overlap itself when wrapped about a skinny leg while still leaving some of the loop fastener material exposed for engagement with the belt-carried hook fastener material.

The hook and loop fasteners are quite long on both sides of the leg wrap, for example loop fastener strips about 7 inches long and hook fastener straps about 9 inches long, so they can be securely wrapped around the entire width of the leg of the animal. The central piece of the leg protective wrap totally encloses the leg of the animal, then the belts are pulled around the leg and fastened, so the animal's leg is encased in the leg wrap. Two tying straps 120, 121 are sewn into one transverse end of the wrap (the top end), and they can be tied around the neck of the animal, attached to the collar, etc. The two neck ties are placed equidistant across the top of the front leg wrap, wrap around the shoulders of the dog and can then be tied around the neck or attached to the collar.

The inner lining of the leg protective wrap is on the side or face of the central piece to be placed against the body of the animal in use of the wrap, and is accordingly made from two layers of cotton flannelette to absorb fluid leakage from wounds and to provide cushioning around the wound. With the leg protective wrap having an outer shell made from polyester fleece and an inner lining made from cotton flannelette, the wrap is designed to withstand multiple cleanings by hand or machine washing and air or machine drying to allow long term and repeated use of the wrap. Compared to use of disposable bandages, the washable reusable wrap is environmentally friendly and can be more cost efficient in use in healing of different wounds over time.

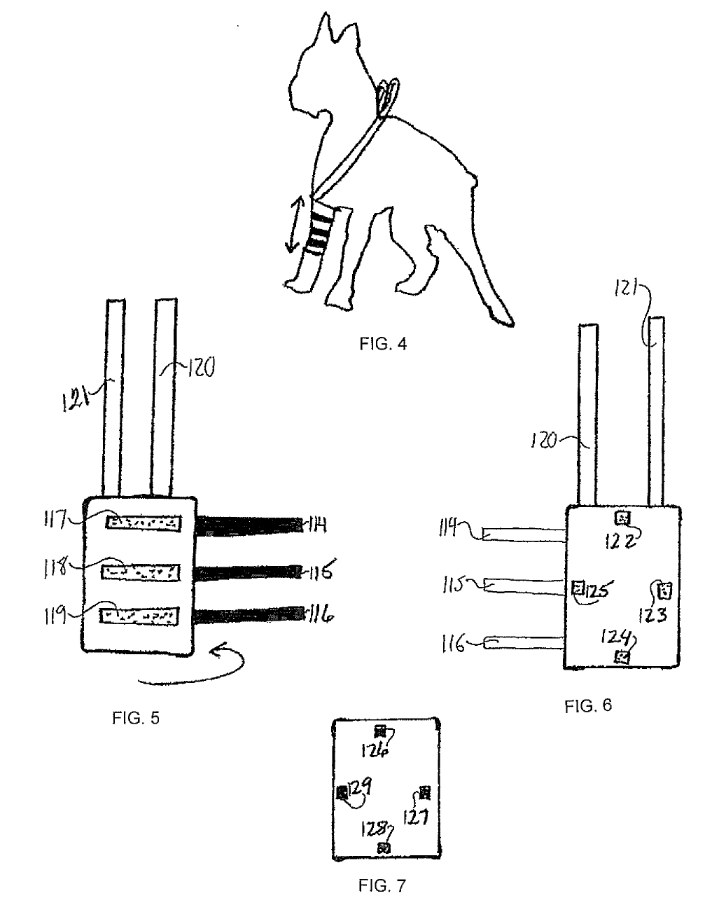

FIG. 4 illustrates a dog wearing the leg wrap on a front leg. The wrap is placed around the leg of the dog, with the person applying the wrap pulling one side of the central piece of the wrap towards them and folding the rest of the wrap around the leg and securing the belts onto the now-wrapped central piece using the hook and loop fasteners.

FIG. 6 shows the lining side of the front leg wrap with two layers of flannelette fabric for absorption and protection. There are four small loop fastener closure pieces 122, 123, 124, 125 sewn into the lining proximate respective sides of the central piece of the wrap, for example each about 1 inch inward from the respective edge of the rectangular piece. The protective leg wrap preferably comes as part of a kit with two removeable cotton pads, each pad preferably made from three layers of cotton flannelette. These pads are preferably rectangular in shape and slightly smaller than the central piece of the wrap, for example having each side of the pad spaced approximately one inch inward from a respective side of the wrap piece when placed centrally thereover. On one side of the pad are sewn four small pieces of hook fastener material which are positioned on the pad adjacent the four sides thereof at generally central positions the sides in order to line up with the fastener material pieces on the central portion of the wrap when the pad is centered thereover. The four loop fastener elements 122, 123, 124, 125 sewn to the lining of the wrap to thus fasten to the corresponding hook fastener elements 126, 127, 128, 129 on the pad to selectively receive and secure the one of the two extra pads on the wrap. Each pad can thus be quickly and easily attached to the main leg wrap to prevent slippage between the wrap and the pad when used on the animal.

### Foot/Paw Protective Wrap System

The protective foot wrap system follows similar design principles. It has an outer shell made from polyester fleece and an inner lining made from two layers of cotton flannelette. As shown in FIGS. 9 and 10, the shape of the foot wrap resembles an elongated dog bootie with a closed-end foot portion opening into a tube-like sock which is to run up a portion of an animal's leg when worn, for example extending about eight inches up an animal's leg. The protective foot wrap is sewn so that one side is open about ¾ of the length of the wrap to facilitate placement of the paw in the wrap, the opening extending from the top of the wrap to about the top of the foot as schematically shown by a broken line in FIG. 9. This creates an opening in the wrap on one side and allows the animal's foot to be quickly placed into the bottom of the wrap.

As shown in FIG. 10, the bottom of the protective foot wrap has a 100% polyester grip pad or stop 40 sewn into the bottom of the foot portion, as defined by the bottommost portion of a rear side of the wrap that extends upward a short distance from the closed bottom end to a point spaced below the bottom end of the side opening, for traction. In addition to the full size inner layer spanning the full surface area of the outer layer to fully cover the interior of the wrap once sewn into the side-slitted sock-like structure of FIG. 9, two smaller pieces of cotton flannelette the same size of the grip pad are sewn onto the inner layer before forming the side-slitted sock-like structure to provide extra cushioning for the animal's foot at a position on the inner side of the portion of the wrap where the grip pad is mounted at the outer side.

There are three hook and loop fasteners sewn into the protective foot wrap, placed equidistance apart along the side seam. A strip of loop material for each of these fasteners is sewn over its entire length onto the front side of the foot wrap. This strip extends from near the open side, for example about one inch inward therefrom, across the front of the wrap toward the opposite closed side. The three loop-carrying straps 33, 34, 35 are sewn equidistant from one another up the sides of the wrap, for example starting about four inches up from the closed bottom end of the wrap. The hook-carrying straps 36, 37, 38 of the hook and loop fasteners are each sewn at one end to the wrap, for example by sewing of this end to the wrap at a position under a respective end of the strip of loop material, so that when pulled in a straight line to project to one side of the wrap, the strap extends parallel to the loop strips spaced apart along the open edge. In use, the hook fastener straps are wrapped around the leg to come back to the loop fastener material for engagement thereon so that the straps circle around the wrap to secure to the loop-carrying strips sewn into the front of the protective foot wrap, and in doing so, effectively close the open side of the wrap. The straps of the fasteners are quite long so they can be securely wrapped around the entire leg of the animal, for example each being about 9 inches long and the loop-carrying strip of each fastener being about 3 inches long.

The protective foot wrap preferably comes with two washable, removeable socks (rather than the pads used in the other two wraps). Each of these sock-like casing or inserts, one of which is shown in FIG. 11, functions as an absorptive padding and is made from three layers of flannelette sewn up the sides to look like socks. This sock slides over the animal's foot and secures on the dog's leg at the top of the sock with a small hook and loop closure. Once the animal's foot is in the sock, and the sock attached to the dog's leg by the closure, the socked foot can be easily placed into the protective foot wrap and the wrap can be closed with the three hook and loop closure belts.

The protective foot wrap has a draw string closure 39 sewn into the wrap a short distance from the top thereof, for example about 1 inch down from the top edge of the wrap that forms the open top end of the wrap's substantially tubular closed-bottom sock-like configuration. This drawstring can be tightened and tied adjacent the top of the wrap, which may be situated about halfway up the animal's leg, to help keep the wrap on the leg even when there is excessive movement.

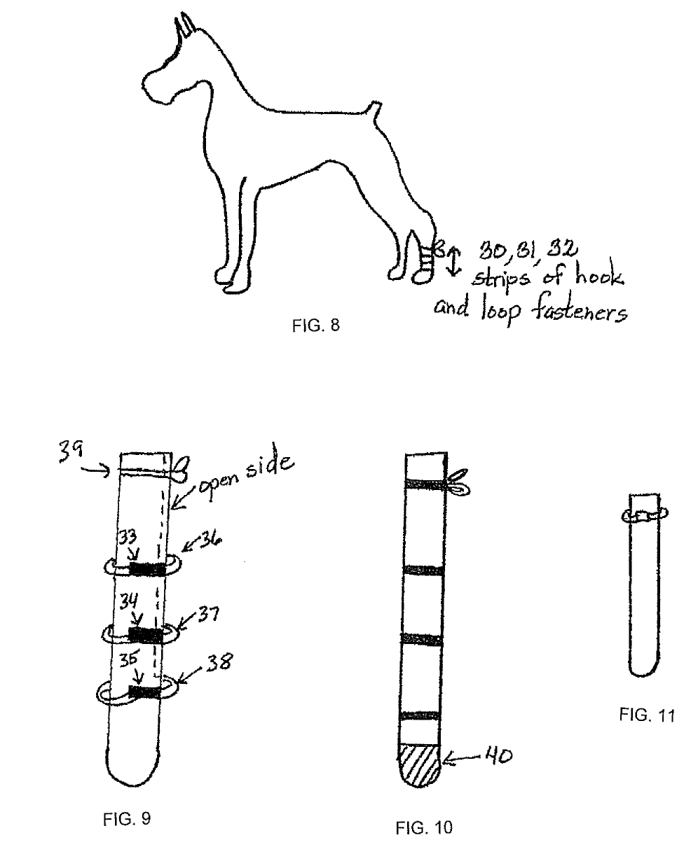

FIG. 8 shows a dog wearing the foot wrap on its back leg. The dog's paw is lowered into the wrap, then the sides of the wrap are brought together and fastened with the three hook and loop fasteners 30, 31, 32 provided by the strips of loop material 33, 34, 35 and mating strips of hook material 36,37,38.

### SUMMARY

Disclosed above are protective wrap systems for small animals to provide coverage for wounds on the torso, upper front legs, and feet. The systems are especially suited for house pets (cats and dogs) for longer term care of wounds in the home setting. The wrap system for the torso includes a rectangular shaped covering of polyester fleece with three hook and loop belts sewn into the covering at spaced positions along the longitudinal axis and two tying straps sewn at spaced positions along one transverse end to wrap and tie around the neck of the animal. Since there is no neck piece, the torso wrap can be used to protect any wound on the torso of the animal (either side, along the back, along the belly) by positioning the wrap over the injury and fastening the belts and ties around the animal. The front leg wrap follows the same basic design, featuring a covering of rectangular shape and made from polyester fleece with three hook and loop belts spaced along the longitudinal axis of the wrap. Two tying straps are attached to one transverse end of the wrap to wrap and tie around the neck of the animal. The foot wrap resembles an elongated “dog bootie” with three hook and loop belts sewn at spaced positions along the longitudinal axis and a drawstring running along the top of the wrap for added security in maintaining its position when worn.

The uniqueness of these wraps is due in part to their inner design which involves a layered lining on the inside of each wrap to provide absorption of fluids from wound leakage. Each of the torso and leg wraps preferably also comes with two layered pads which fasten into the wraps with small hook and loop fasteners for extra protection when wounds are fresh. The foot wrap has a protective sock (instead of a pad) made from absorptive fabric which the owner slides up the foot and leg of the animal, then the foot is then placed in the actual wrap. All three wraps have been designed to handle washing on a daily basis, the pads and the wraps can be easily washed/dried and reused hundreds of times. The inclusion of an absorptive lining as part of the wrap and the removable washable liners create a progressive system for the owner/veterinarian which can be adapted to wound care management over various stages of the healing process. One can use the pads and the lined wrap for the initial stages of an injury when there is the most bleeding and drainage, then move on to just using the wrap with the built in lining as the wound heals and requires less coverage.

### Advantages

The present invention furthers development in this field over the prior art by:

1. Providing a progressive system of wound care for the owner/veterinarian in one wrap kit offering multiple uses and choices depending upon the severity and healing stage of the injury. All three styles of wrap have built in liners which can be used in conjunction with a veterinarian's wound dressing to secure the dressing and protect the area. Any wound dressings can be eliminated and the removeable, absorbent cotton pads can be secured into the wraps, providing absorption for fluid leakage, and padding around the wound. Drains can hang freely within the wrap and drain directly into the padding. As wounds heal the pet owner can stop using the absorbent pads and use the wrap as a protective covering because it has a built-in, lighter absorptive lining. In other words, the attachable pads can be inserted in the early stages of an injury to provide maximum absorption of fluids and leakage from wounds. As the wound heals and requires less coverage, the wraps can be used without the attachable pads, as they still have a thinner absorptive lining sewn into the wrap. The inclusion of three belts using strips of hook and loop fastener elements on each style of wrap allows the pet owner to vary the pressure across the affected area by individually adjusting the tension on each belt fastener to provide less/more pressure around the wound site.

2. The entire wrap system has been designed to handle repeated or regular washing, for example daily washing/drying in home machines. This makes wound care for the pet owner easy, convenient, and very inexpensive. The wraps are currently being marketed with two absorbent pads in each package, so the owner can wash one pad while using another in the wrap. This means a one-time purchase of an easy care, reuseable, washable, protective system for the pet owner which can be used for extended periods of time, reaching many years in most cases.

3. The choice of fabrics for the wraps and pads of the preferred embodiments (polyester fleece and cotton flannelette) have proven to be very appealing to pet owners and pets. The fabric is strong, yet very soft and pliable. It is tolerated very well by animals because it is soft and comforting, molding to body shape. Both fabrics wash and wear very well. Also, manufacturing the wraps out of “fun fleece patterns” appears to be especially appealing to many pet owners (the wraps do not look like bandages).

4. The wrap system defines the bandage. It doesn't have to be used in conjunction with any other separately acquired attachments or supplies.

5. All three styles of wrap are very easy and quick to put on and remove from an animal. They have all been field-tested and adjusted to meet veterinarian and pet owner concerns.

### Alternatives

The illustrated embodiments use belt configurations in which each belt features a strap having either a hook or loop fastener element thereon to couple with a respective mating fastener element on either the wrap itself or a corresponding strap extending from or about the wrap in an opposite direction. It will be appreciated that other types of fasteners may be employed, for example using safety pins or spaced apart snap or button elements, but hook and loop fasteners provide a much preferred combination of safety (no sharp points), adjustability (infinite attachment points along a continuous length of fastening material), security (permanent attachment of fastener components to the wrap prevents loss) and ease of attachment and detachment. Likewise, other fastening mechanisms may be used in the selective attachment of the pads to the wraps, but again hook and loop fasteners are advantageous.

Each belt in the illustrated torso wrap embodiment uses a pair of straps fixed to the wrap at opposite sides thereof to fasten together at a position opposite the wrap in a loop defined by the straps and the wrap to close around the animal's torso, while each belt in the illustrated leg wrap embodiment uses a single strap fixed to one side thereof to fasten a distal end of the strap onto the wrap from an opposite side thereof to close a loop formed by the wrap and strap around the animal's leg. The torso wrap could instead use a single-strap belt configuration in which the single strap of each belt is long enough to reach fully about the torso to connect the distal end of the strap to the wrap. Also, in single strap arrangements in which the strap is long enough, the strap may carry opposing ones of the loop and hook elements on its opposing faces in order to fasten onto itself after extending fully around the body part on which the wrap is placed.

It will be appreciated that materials other than those explicitly disclosed herein above may be used to produce a wrap with a similar combination of a durable, machine washable cover layer and a more absorbent liner layer. For example, wraps have been produced with denim, cotton and polyester coverings and the cotton flannelette liner used in the detailed embodiments. Of these materials used to date, the polyester fleece has been found to best mold to the body shapes of animals. However, the choice of material may be influenced by a particular intended end use. For example, a denim wrap was produced for use on a calf to avoid straw from sticking to a fleece covering. This also demonstrates that the wrap is not necessarily restricted to use on household pets, and may be applicable for farm or barnyard animals, livestock or other domesticated animals.

It will be appreciated that wraps similar to those disclosed herein may be used or altered on other body parts. For example, a wrap similar to the torso wrap may be altered to lack neck straps and be altered in dimension for use as a tail wrap suitable for use on an injured tail of a dog or other animal. Similarly, a neck wrap may use a rectangular or otherwise shaped wrap dimensioned for wrapping about an animal's neck and having side-projecting straps suitable for closing about the animal's neck to support the wrap thereon.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A wound wrap system for an animal comprising:
a flexible wrap comprising a cover layer of a first material and a liner layer fixed to the cover layer and comprising an absorbent material different than the first material;
a set of flexible straps each having a first connection to the flexible wrap and each having a distal end spaced from and movable relative to the first connection, each strap carrying a fastening element thereon between the first connection and the distal end, each fastening element being selectively fastenable with a mating fastening element carried on the flexible wrap to close a loop around a body part of the animal to secure the absorbent material of the liner layer of the flexible wrap over a wounded area of the body part.

2. The wound wrap system according to claim 1 each flexible strap extends from the wrap at a side edge thereof and the wound wrap system further comprises an additional strap arrangement extending from the wrap at an end edge thereof so that the flexible straps and the additional strap arrangement project from the wrap along opposing ones of perpendicular axes in a plane of the wrap when laid out flat.

3. The wound wrap system according to claim 2 wherein the wrap is sized to wrap about at least a portion of an animal's torso, each flexible strap being sufficiently long to close the loop around the animal's torso and the additional strap arrangement being sufficiently long to secure about an animal's neck.

4. The wound wrap system according to claim 2 wherein the additional strap arrangement comprises a pair of straps extending from the end edge of the wrap.

5. The wound wrap system according to claim 1 wherein the wrap is sized to wrap about at least a portion of an animal's leg, each flexible strap being sufficiently long to close the loop around the animal's leg.

6. The wound wrap system according to claim 2 wherein the wrap is sized to wrap about at least a portion of an animal's leg, each flexible strap being sufficiently long to close the loop around the animal's leg and the additional strap arrangement being sufficiently long to secure about an animal's neck.

7. The wound wrap system according to claim 1 wherein the wrap is configured into an elongated substantially tubular configuration having an open end and an opposing closed end, with the liner layer of the absorbent material disposed inside the substantially tubular configuration.

8. The wound wrap system according to claim 7 wherein an longitudinal opening in one side of the substantially tubular configuration extends from the open end thereof toward, without reaching, the opposing closed end, a portion of the substantially tubular configuration along which the longitudinal opening extends defining a leg portion for receipt of an animal's leg and a remaining portion of the substantially tubular configuration between the longitudinal opening and the closed end defining a foot portion for receipt of an animal's foot.

9. The wound wrap system according to claim 8 wherein at least some of the flexible straps are connected to the substantially tubular configuration at positions along the leg portion thereof.

10. The wound wrap system according to claim 7 comprising a drawstring disposed about the substantially tubular configuration of the wrap adjacent the open end thereof and operable to reduce a size of the open end.

11. The wound wrap system according to claim 7 comprising a gripping element situated externally on the tubular configuration of the wrap adjacent the closed end thereof.

12. The wound wrap system according to claim 7 comprising an absorbent tubular insert sized for receipt within the substantially tubular configuration of the wrap and being open at one end and closed at an opposing end.

13. The wound wrap system according to claim 12 wherein the absorbent tubular liner is made of the same absorbent material as the liner layer of the wrap.

14. The wound wrap system according to claim 1 comprising at least one additional absorbent layer between the cover layer and the liner layer.

15. The wound wrap system according to claim 14 wherein the at least one additional absorbent layer comprises the same absorbent material as the liner layer.

16. The wound wrap system according to claim 1 comprising at least one absorbent pad of similar shape and size to the wrap and selectively attachable and detachable to the liner layer of the wound wrap system.

17. The wound wrap system according to claim 1 wherein the liner layer comprises cotton flannelette.

18. A method of animal wound care comprising the steps of:
(a) providing a wound wrap system comprising:
a flexible wrap comprising at least one layer of absorbent material;
a set of flexible straps each extendable from a side edge of the wrap; and
an additional strap arrangement extending from the wrap at an end edge thereof so that the flexible straps and the additional strap arrangement project from the wrap along opposing ones of perpendicular axes in a plane of the wrap when laid out flat;
(b) using the flexible strap to close loops around a torso or front leg of an animal to secure the absorbent material of the flexible wrap over a wounded area of the torso or front leg; and
(c) extending portions of the additional strap arrangement about opposite sides of a neck of the animal to interconnect the portions of the additional strap arrangement across a back of the neck to provide additional support of the wrap on the torso or front leg.

19. A method of animal wound care comprising the steps of:
(a) providing a wound wrap system comprising:
a flexible wrap comprising a cover layer of a first material and a liner layer fixed to the cover layer and comprising an absorbent material different than the first material; and
an absorbent pad of similar shape and size to the wrap and selectively securable against the liner layer of the wound wrap system;
(b) with the absorbent pad secured against the liner layer of the wound wrap system, securing the wrap about at least a portion of a body part of the animal with the absorbent pad facing the body part and covering a wounded area thereof;
(c) keeping the wrap and the absorbent pad on the animal for a period of time with the wounded area being protected by the wrap and the absorbent pad during said period of time;
(d) removing the wrap from the animal and removing the absorbent pad from the wrap;
(e) securing the wrap at least partially about the body part of the animal with the liner layer of the wrap facing toward the body part to again cover the wounded area; and
(f) washing the absorbent pad while the wrap is worn by the animal to prepare the absorbent pad for later re-use if needed.

20. The method according to claim 18 wherein the wound wrap system further comprises an additional absorbent pad other than that removed in step (d), but also of similar shape and size to the wrap and selectively securable against the liner layer of the wound wrap system, and depending on a desired degree of absorbency based on a status of the wound discovered in step (d), step (e) is carried out either (i) with the additional absorbent pad secured against the liner layer of the wrap or (ii) with no separate absorbent pad secured against the wrap so that the absorbent layer of the wrap is placed directly over the wounded area of the animal.
